(19) United States
(12) Patent Application Publication
(10) Pub. No.: US 2010/0144019 A1
(43) Pub. Date: Jun. 10, 2010

(54) Photobioreactor

(76) Inventor: Chao-Hung Hsu, Taipei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/314,167

(22) Filed: Dec. 5, 2008

(51) Int. Cl. C12M 1/36; C12M 3/00

(52) U.S. Cl. 435/286.1; 435/292.1

(57) ABSTRACT

A photobioreactor used for microalgal culture consists a main body of bioreactor, a plurality of built-in light sources, a mechanical stirring apparatus and a pneumatic mixing device. The main body of the bioreactor is a hollow chamber and the plurality of built-in light sources connects the chamber from outside to inside for light distribution. The mechanical stirring apparatus is disposed in the chamber to generate liquid circulation. The pneumatic mixing system generates bubbles to suspend microalgae in the culture medium.

### BACKGROUND OF THE INVENTION

1. Field of the Invention

The present invention relates to a photobioreactor, and more particularly to a photobioreactor for cultures of microalgae.

2. Description of Related Art

Microalgae are one kind of unicellular organism that, using photosynthesis, can convert solar energy into fat and stored in cell bodies. It is the most potential biomass material for next generation biofuel application. Microalgae have a high nutrition value and can thus be turned into various nutritious supplements. Due to their high economic value, more and more scientists have commenced the study on microalgae culturing technology to reduce their production costs and improve their production efficiency.

FIG. 1 illustrates a conventional open pond culturing approach which includes a plurality of ponds 10. Each pond 10 has a robot arm 12. Since the conventional approach cultivates microalgae in an open way, performing photosynthesis requires large light receiving area and low water depth to obtain enough sunlight. Moreover, microalgae on the bottom of the pond 10 cannot fully grow because lacking of sunlight. Periodically stirring by the robot arm 12 is necessary in FIG. 1 design.

FIG. 2 illustrates a conventional closed system for cultivating microalgae. It includes a pipeline 20 with a water inlet 21 and a water outlet 22. The system must have longer pipe length and smaller pipe diameter. The best design for the pipe is U-shape, which can provide longer length and save space. Small diameter pipe results in the small culture volume, however, large diameter pipe causes poor sunlight exposure to microalgae. The pneumatic mixing apparatus (not shown) is beneficial to this system.

Accordingly, the conventional open pond or the conventional closed pipeline system for cultivating microalgae cannot provide both sufficient sunlight environment and large culture volume, simultaneously.

Hence, there is a need of novel photobioreactor which can overcome the above described shortcomings and provide sufficient and uniform sunlight for cultures of microalgae, thereby reducing the culturing costs and improving the production efficiency.

### SUMMARY OF THE INVENTION

A main object of the present invention is to create a novel photobioreactor which provides sufficient and uniform light sources for microalgal culture.

To achieve the above-mentioned object, the photobioreactor includes a bioreactor main body, a plurality of built-in light sources, a mechanical stirring apparatus, a pneumatic mixing device, a temperature control device and a pH-value control device.

The bioreactor main body is a hollow chamber and has an air inlet, an air outlet, a water inlet and a water outlet. Water and microalgae may be put into the bioreactor main body through the water inlet. The built-in light sources extend from outside to inside the bioreactor main body and emit light inside the bioreactor main body. The mechanical stirring apparatus is disposed in the bioreactor main body to generate circular liquid movement via rotating blades. The pneumatic mixing device is connected with the air inlet and may stir the liquid in the bioreactor main body by generating bubbles. The temperature control device and pH-value control device are used for controlling the temperature and pH-value of the liquid in the bioreactor main body, respectively. The optimum growth environment for microalgae can therefore be achieved.

Each of the built-in light sources includes a light receiving end, a total reflection tube and a light emitting end. The light receiving end and the light emitting end are arranged on the two ends of the total reflection tube, wherein the light receiving end is located outside the bioreactor main body and the light emitting end is located inside the bioreactor main body. The light receiving end includes a light inductor for detecting the irradiation direction of an external light source and a driving device which is used for driving the light receiving end to point into the irradiation direction of the external light source. The inside of the total reflection tube is made of a material with high reflectivity and the light emitting end is made of a transparent material. For avoiding attachment of microalgae, the inner surface of the bioreactor main body and the outer surface of the built-in light source are all processed by surface treatment technology.

Furthermore, the photobioreactor of present invention includes a solar energy electricity device and a control device which monitors internal light intensity and controls the solar energy electricity device to emit light to inside of the bioreactor main body during insufficient lighting condition. The solar energy electricity device may also be replaced by a wind energy electricity device which emits light to inside of the bioreactor main body during insufficient lighting condition.

Accordingly, the photobioreactor of present invention has a plurality of build-in light sources for providing sufficient light to the bioreactor main body. As a result, the photobioreactor of present invention can culture the larger amount of microalgae so as to reduce the culturing costs.

To further understand the advantages and spirits of present invention, please refer the following detailed descriptions and drawings.

### BRIEF DESCRIPTION OF THE DRAWINGS

FIG. 1 is a schematic view of a conventional open pond for microalgae culturing;

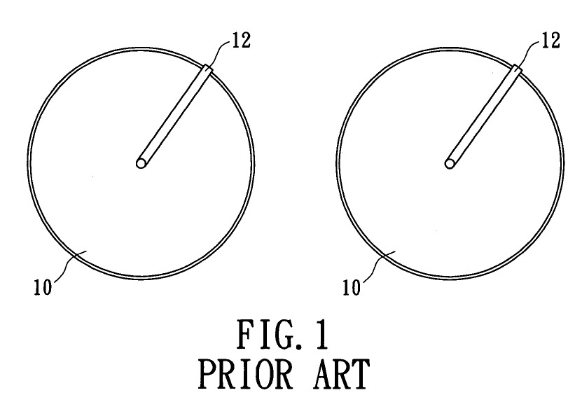

FIG. 2 is a schematic view of a conventional closed pipeline system for microalgae culturing;

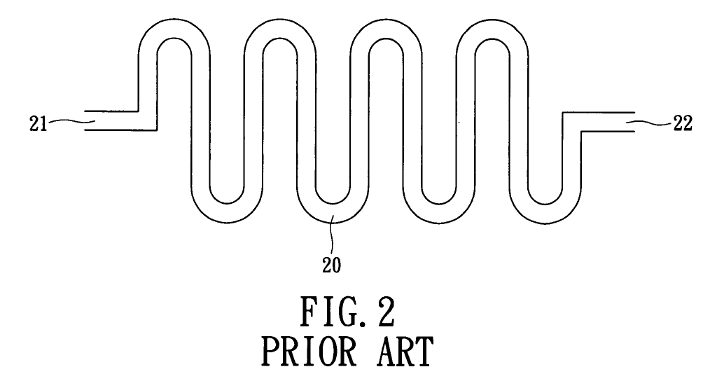

FIG. 3 is a schematic view of a photobioreactor of present invention;

FIG. 4 is a schematic view of a build-in light source of present invention; and

FIG. 5 is a schematic view showing that present invention uses a computer to control the photobioreactor system.

### DETAILED DESCRIPTION OF PREFERRED EMBODIMENTS

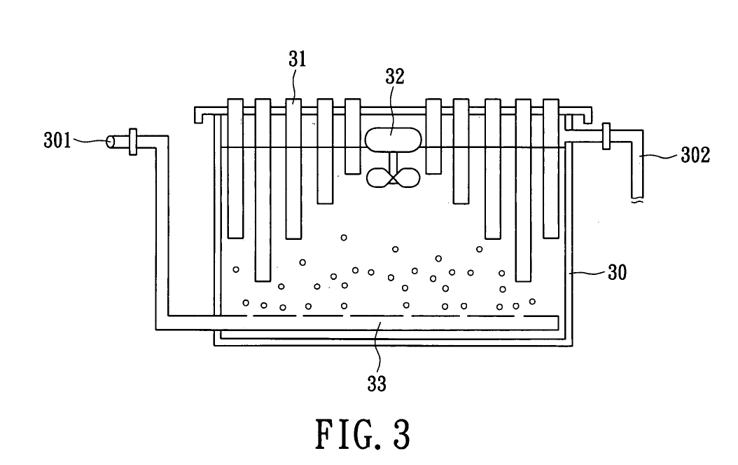

FIG. 3 illustrates a photobioreactor in accordance with the present invention. The photobioreactor includes a bioreactor main body 30, a plurality of built-in light sources 31, a mechanical stirring apparatus 32, a pneumatic mixing device 33, a temperature control device and a pH-value control device (not shown).

The bioreactor main body 30 is a hollow chamber which is preferably cylindrical. The bioreactor main body 30 has an air inlet 301, an air outlet 302, a water inlet and a water outlet (not shown). Culture medium and microalgae may be put into the bioreactor main body 30 through the water inlet, and gases such as carbon dioxide may be put into the bioreactor main body 30 through the air inlet 301. For improving growth rate of the microalgae and reducing costs, the present invention may also combine a plurality of bioreactor main bodies 30 in series.

The number of built-in light sources 31 is determined by the volume of the bioreactor main body 30 and the light intensity provided by built-in light sources. In order to optimize sufficient lighting condition in bioreactor main body 30, the built-in light sources 31 are extended from outside to inside the bioreactor main body 30 and emit light inside the bioreactor main body 30. Furthermore, the built-in light sources 31 may have different lengths, which is determined according to actual demands.

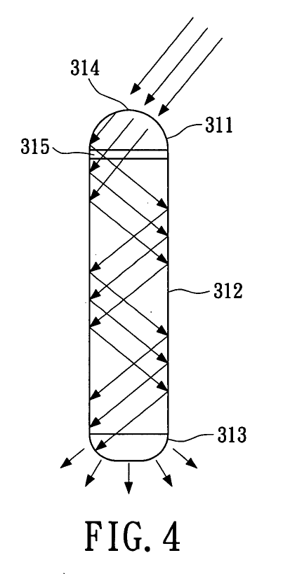

FIG. 4 indicates a built-in light source 31. It includes a light receiving end 311, a total reflection tube 312 and a light emitting end 313. The light receiving end 311 and the light emitting end 313 are arranged on the two ends of the total reflection tube 312, wherein the light receiving end 311 is located outside the bioreactor main body 30 and the light emitting end 313 is located inside the bioreactor main body 30. The light receiving end 311 includes a light inductor 314 for detecting the irradiation direction of an external light source and a driving device 315 which is used for driving the light receiving end 311 to point into the irradiation direction of the external light source so as to obtain the optimum light intensity. The inside of the total reflection tube 312 is made of a material with high reflectivity and the light emitting end 313 is made of a transparent material. Additionally, the inner surface of the bioreactor main body 30 and the outer surface of the built-in light source 31 are all processed by surface treatment technology, thereby avoiding attachment of the microalgae.

In FIG. 3, the mechanical stirring apparatus 32 is disposed in the bioreactor main body 30 to generate liquid circular movement in the bioreactor main body 30 via rotating blades. The pneumatic mixing device 33 is connected with the air inlet 301 and generates bubbles in bioreactor main body 30. Both provide mixing function to suspend microalgae uniformly. The temperature control device is used for controlling the medium temperature to reach optimum growth temperature in the bioreactor main body 30. The pH-value control device is used to detect the pH-value of the culture medium in the bioreactor main body 30. Adjusting the pH-value of medium is necessary to achieve optimum growth environment.

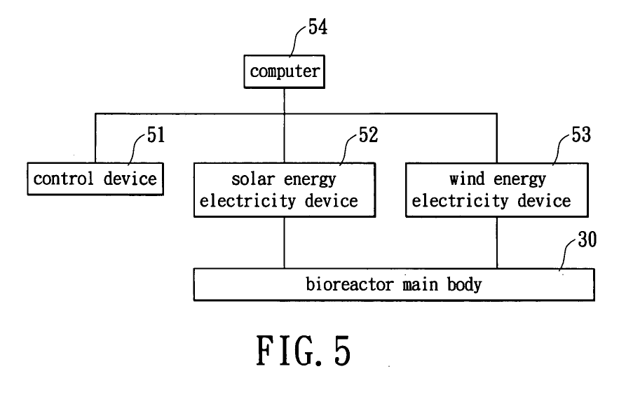

In order to avoid microalgae growth affected by climate and day-night change, solar energy electricity device 52 and wind energy electricity device 53 are designed to maintain the stable light source in the photobioreactor system (FIG. 5). The present invention includes a solar energy electricity device 52 and a control device 51 which monitors internal light intensity and controls the solar energy electricity device 52 to emit light to inside of the bioreactor main body 30 during insufficient lighting condition. Further, the solar energy electricity device 52 may be replaced by a wind energy electricity device 53 or combined with a wind energy electricity device 53. When the internal light intensity isn't enough, the solar energy electricity device 52 or/and the wind energy electricity device 53 emit light to the inside of the bioreactor main body 30. The above described three devices are controlled by a computer 54. Additionally, the solar energy electricity device 52 and the wind energy electricity device 53 may apply LED light source which can be set to emit light within specific wavelength range, according to the species of microalgae cultivated in bioreactor main body 30 for acceleration of microalgae growth.

Accordingly, the photobioreactor of the present invention has a plurality of build-in light sources 31 to provide sufficient light into the bioreactor main body 30, thereby the photobioreactor of the present invention can culture the larger amount of microalgae so as to reduce the culturing costs.

Above disclosed are only the specification and the drawings of the preferred embodiment of the present invention. It is therefore not intended that the present invention is limited to the particular embodiments disclosed. It will be understood by those skilled in the art that various equivalent changes may be made depending on the specification and the drawings of the present invention without departing from the scope of the present invention.

What is claimed is:

1. A photobioreactor, comprising:
a bioreactor main body, being a hollow chamber and having an air inlet and an air outlet;
a plurality of built-in light sources, each light source extending from outside to inside the bioreactor main body;
a mechanical stirring apparatus, disposed in the bioreactor main body for stirring liquid in the bioreactor main body; and
a pneumatic mixing device, connected to the air inlet and stirring the liquid in the bioreactor main body by generating bubbles.

2. The photobioreactor as in claim 1, wherein the bioreactor main body is made of a corrosion resistant material and processed by surface treatment technology for avoiding attachment of microalgae.

3. The photobioreactor as in claim 1, wherein each built-in light source includes a light receiving end, a total reflection tube and a light emitting end, with the light receiving end and the light emitting end being arranged on the two ends of the total reflection tube, the light receiving end being located outside the bioreactor main body and the light emitting end being located inside the bioreactor main body.

4. The photobioreactor as in claim 3, wherein the light receiving end includes a light inductor for detecting the irradiation direction of an external light source and a driving device for driving the light receiving end to point into the irradiation direction of the external light source.

5. The photobioreactor as in claim 3, wherein an inside surface of the total reflection tube is made of a material with high reflectivity and an outside surface of the total reflection tube is processed by surface treatment technology for avoiding attachment of microalgae.

6. The photobioreactor as in claim 3, wherein the light emitting end is made of a transparent material and processed by surface treatment technology for emitting uniform light and for avoiding attachment of microalgae.

7. The photobioreactor as in claim 1, wherein the mechanical stirring apparatus is a blade disposed in the bioreactor main body.

8. The photobioreactor as in claim 1, further comprising a solar energy electricity device and a control device which monitors internal light intensity and controls the solar energy electricity device to emit light to the inside of the bioreactor main body when there is insufficient lighting condition inside.

9. The photobioreactor as in claim 8, wherein the solar energy electricity device apply LED resource which emits light within specific wavelength range according to the microalgae species in the bioreactor main body.

10. The photobioreactor as in claim 1, further comprising a wind energy electricity device and a control device for monitoring internal light intensity and controlling the solar energy electricity device to emit light to the inside of the bioreactor main body when there is insufficient lighting condition inside.

11. The photobioreactor as in claim 10, wherein the wind energy electricity device apply LED resource which emits light within specific wavelength range according to the microalgae species in the bioreactor main body.

12. The photobioreactor as in claim 1, further comprising a temperature control device for controlling the temperature of the liquid in the bioreactor main body and a pH-value control device for detecting the pH-value of the liquid in the bioreactor main body.
